(19) United States
(12) Patent Application Publication
(10) Pub. No.: US 2004/0219589 A1
(43) Pub. Date: Nov. 4, 2004

(54) Ligand profiling and identification

(76) Inventors: Taehoon Lee, Pohang (KR); Joseph Kwon, Pohang (KR); Moon C. Baek, Pohang (KR); Youndong Kim, Pohang (KR); Byoung D. Lee, Pohang (KR); Jong I. Kim, Pohang (KR); Yoe-Sik Bae, Busan (KR); Pann-Ghill Suh, Pohang (KR); Sung Ho Ryu, Pohang (KR)

Correspondence Address: Joseph Hyosuk Kim, Ph.D., JHK Law, P.O. Box 1078, La Canada, CA, 91012-1078, US

(21) Appl. No.: 10/835,922

(22) Filed: Apr. 29, 2004

Related U.S. Application Data
(60) Provisional application No. 60/466,488, filed on Apr. 29, 2003.

(51) Int. Cl. C12Q 1/68; G01N 33/53; G01N 33/537; G01N 33/543

(52) U.S. Cl. 435/6; 435/7.92

(57) ABSTRACT

The present application discloses a method of identifying an active agent in a sample, which steps include subjecting the sample to a plurality of separation principles in parallel, obtaining an active fraction from each separation principle, and profiling physiochemical properties of the active fraction so as to obtain the agent that is common in each of the fractions, thereby identifying the agent of interest.

### CROSS-REFERENCE To RELATED APPLICATIONS

The present application claims the benefit of priority to U.S. Provisional Application 60/466,488, filed Apr. 29, 2003, which is incorporated by reference herein in its entirety.

### BACKGROUND OF THE INVENTION

1. Field of the Invention

The present invention relates to methods for identifying agents having specific activity from a sample. More specifically, the invention relates to the construction of a mixture of active agents such as bio-molecules in library-format by parallel separation processes, which is used to identify functional agents.

2. General Background and State of the Art

Proteomic technology is one of the hottest fields in life science in post-genomic era (Vachet et al.), and requires separation and detection techniques with high resolution to identify novel endogenous protein or peptides. Many instrumental strategies have been developed which are based on efficient separation followed by mass spectrometry identification and quantification. The most commonly accommodated approach in proteomic studies is to separate and visualize as many proteins as possible by two-dimensional electrophoresis, to analyze the expression profile of proteins in a given context, and to subsequently identify differentially expressed proteins by mass spectrometric techniques. However, there are several constraints in utilizing this technique for identifying functionally important proteins or peptides. This procedure heavily relies on the visualization of the proteins by several staining methods for identifying differentially expressed proteins. One of the major constraints of this technology is that proteins having molecular mass in the range of lower than 20 kDa where a lot of medically and commercially important biological ligands such as peptide hormone, neurotransmitters, and G protein coupled receptor ligands are found, are generally not analyzed. Moreover, function-based analysis of the proteins is not allowed by the use of this technique.

Peptide ligands such as peptide hormones, neurotransmitters, chemokines, and cytokines play important roles in many regulatory processes in an organism. These peptides (small polypeptide with a molecular mass of less than 20 kDa) execute a variety of essential functions for the communication between the cells. In terms of medicine, these peptides have been used to diagnose and treat many human diseases. Insulin is an example of a peptide-drug, which can control glucose level in human body and thus has been used as an agent for treatment of diabetes. Other examples of peptide drugs are growth hormone (25 kDa), INF-alpha (19.4 kDa), EPO (18.4 kDa), GM-CSF (13.5 kDa), insulin (5.8 kDa) and so on. Several other peptide drugs such as Glucagon-like peptide 1 (GLP-1), calcitonin, GnRH, parathyroid hormone (PTH), or leptin are in clinical development.

To date, many peptide ligands have been identified from vertebrate and invertebrate sources through labor-intensive and time-consuming purification procedure using sequential column chromatographic separation combined with functional activity assay. Along with the emergence of very sensitive mass spectrometry apparatus and very accurate and miniaturized separation techniques, the trend of the experimental approaches dealing with peptides is changing from one-target-at-one-experiment model to a more comprehensive pattern, designated as peptidomics in analogy with proteomics, enabling researcher to analyze the entire peptidome in a given context at the amino acid sequence level, starting from very little amount of material.

A multidimensional combination of HPLC-HPLC or HPLC-CE on-line or off-line with a mass spectrometer for protein identification has been introduced. Recently, various mass spectrometry systems have been developed and applied to identify and to characterize relevant proteins from crude mixtures, which is useful for the initial stages of purification and separation. Additionally, MS-MS analysis instead of Edman degradation peptide sequencing has been used. By the help of the development of sensitive analysis tools, peptidomics overcomes the drawback associated with dealing with small amounts of bio-molecules, which hampered traditional purification efforts.

In addition, function-based, but not differential expression-based, identification of useful biological ligands is possible by the integration of expression peptidomic approach with functional profiling of endogenous ligand libraries. The ligand libraries can be prepared by using parallel separation processes such as column chromatographic separation methods using any source of starting materials, and the biological activity of any given set of ligand libraries can be measured by the use of a variety of assays including newly developed cell-based activity assay methods. The bio-molecules in the active fractions from each column chromatography that exhibit certain physicochemical properties may be responsible for the measured biological activities in the fractions as well. This kind of integration technology, so called Ligand Profiling and Identification (LPI) technology can be utilized for identifying novel peptide ligands having different biological activities, as the pluralities of sets of endogenous bio-molecules is constructed in ready-to-use library format, and all the data produced can be stored in a database. This strategy can be applied for the analysis of diverse groups of molecules such as carbohydrates, lipids, nucleic acids, proteins and peptides, or any other molecule having any structure.

### SUMMARY OF THE INVENTION

The present invention is directed to a method of profiling and identifying a bio-molecule agent, such as a ligand, comprising using parallel molecule separation methods, which use different separation principles, combined with at least one functional assay and molecule mass detection methods such as mass spectrometry for identifying the agent or bio-molecule present in any type of sample, which agent is preferably novel.

In one aspect of the invention, this method may include four components. First, crude sample extract or biochemically (eg. chromatographically) enriched preparation is applied to various separation methods having different separation principles such as various types of chromatographies, and the fractions eluted from each separation principle are constructed in a library-format. Second, one or more fractions showing a specific activity profile of interest from each the fractions is/are selected from the library. A typical separation method may utilize column chromatography. One or more active or bio-active agent can be located at the specified fractions from each column. Third, the separate mixture of active agent or bio-molecules from each active fraction is separately analyzed by liquid column chromatography combined with mass spectrometer. Fourth, the structure of the active agent of interest is determined by a tandem mass spectrometer.

The invention is directed to a method of identifying an active agent in a sample comprising subjecting the sample to a plurality of separation principles in parallel, obtaining an active fraction from each separation principle, profiling physiochemical properties of the active fraction so as to obtain the agent that is common in each of said fraction, wherein said agent is identified. In this regard, the sample may be from any source at all, including environmental, industrial, biological, so long as an assay exists to be able to distinguish and separate the active agent from the other molecules in the sample. The assay may be an enzymatic assay, cell-based assay, physical assay or any chemical assay. And the starting sample may be for example, a mammalian extracellular fluids, mammalian cell/organ extract, plant cell/organ extract and so on.

In the method above, the active agent may be a bio-molecule such as without limitation polypeptide, peptide, carbohydrate, lipid, or nucleic acid. Such bio-molecule or ligand may be a factor that binds to a G protein coupled receptor, that specifically binds to a growth factor receptor, or that specifically binds to an adhesion molecule. The bio-molecule may also be without limitation a protease.

In one aspect of the invention, the present invention is directed to identifying a small active agent, in particular, a small bio-molecule. The small bio-molecule may be less than about 20 kDa, 10 kDa, or 2 kDa.

The separation principle, which is directed to the various physical or chemical properties that are taken advantage of in separating the active agent from others that may be used in the invention include use of various chromatography apparati, and may include without limitation column chromatography methods, which in turn may include without limitation hydrophobic interaction chromatography, cation exchange chromatography, anion exchange chromatography, gel permeation chromatography, or affinity chromatography. Other types of separation procedures may also be used.

The profiling of the fractions based on physiochemical properties may include passing the active fraction through a liquid chromatography (LC) column, and further to an element such that a 2-dimensional separation of the active agent may be observed. In one aspect, such profiling may occur through a virtual 2-dimensional technique, which may be facilitated by without limitation mass spectrometer methods. In this aspect, the liquid chromatography column that is used may be nano-LC column, and the LC column may be connected to a mass spectrometer.

In another aspect of the invention, the invention is directed to instructions that describe how to use the ligand profiling and identification as described above, for example which may be described in an instrument manual or a catalog.

In another aspect, the invention is directed to a method of assigning a code to an active agent containing fraction in a sample comprising subjecting the sample to a plurality of separation principles in parallel, obtaining an active fraction from each separation principle, assigning a first unique identifying code to the active fraction, and gathering and combining the identifying code to form a second unique assigning code for the active agent containing fraction. The fractions obtained from parallel processing may comprise a ligand library, from which the active fractions may be obtained.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows three-dimensional concept of Ligand Profiling and Identification (LPI) technology. Any active agent can be assumed to be present as a single dot in three dimensional space composed of three axes of functional activity, ligand library, and physicochemical property. Bio-molecules presented as a single dot can be identified by tandem mass spectrometric analysis.

FIG. 2 represents LPI technology composed of 4 different modules: Ligand Library, Functional Activity Profiling, Physicochemical Profiling, and Identification.

FIG. 3 shows the theoretical background by which the technical advantage of using parallel processing over sequential purification can be explained in terms of yield.

FIG. 4 explains how the components of each module comprising LPI technology can be integrated for identifying specific class of active bio-molecules. The abbreviations used in the drawing are represented as follows: E-PepLibrary, endogenous peptide library; E-ProLibrary, endogenous protein library; E-LipidLibrary, endogenous Lipid library; E-CarboLibrary, endogenous carbohydrate library; E-MetaLibrary, endogenous Metabolite library; CSRA, cell-based signaling response assay; EAA, enzyme activity assay; BIA, bio-molecular interaction assay; FHSA, Fluorescence-based high sensitivity assay; PepLPI, peptide ligand profiling and identification; ProLPI, protein ligand profiling and identification.

FIG. 5 describes detailed flowchart for identifying novel ligands involved in the regulation of glucose metabolism from human fat.

FIG. 6 shows LC/MS analysis of the active fractions from human fat & display of bio-molecules in virtual 2D-space.

FIG. 7 shows variations of the LPI technology by adding the combination of sequential and parallel separation method, a protease scanning, and the direct comparision of amino acid sequence information obtained from tandem mass spectrometry.

FIG. 8 shows an example of successful application of protease scanning method in excluding unwanted contaminats from the bio-molecules of interest.

### DETAILED DESCRIPTION OF THE PREFERRED EMBODIMENTS

In the present application, “a” and “an” are used to refer to both single and a plurality of objects.

Ligand Profiling and Identification (LPI)

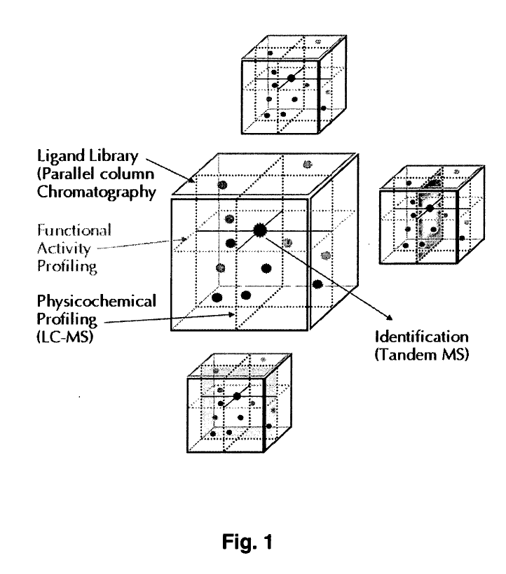

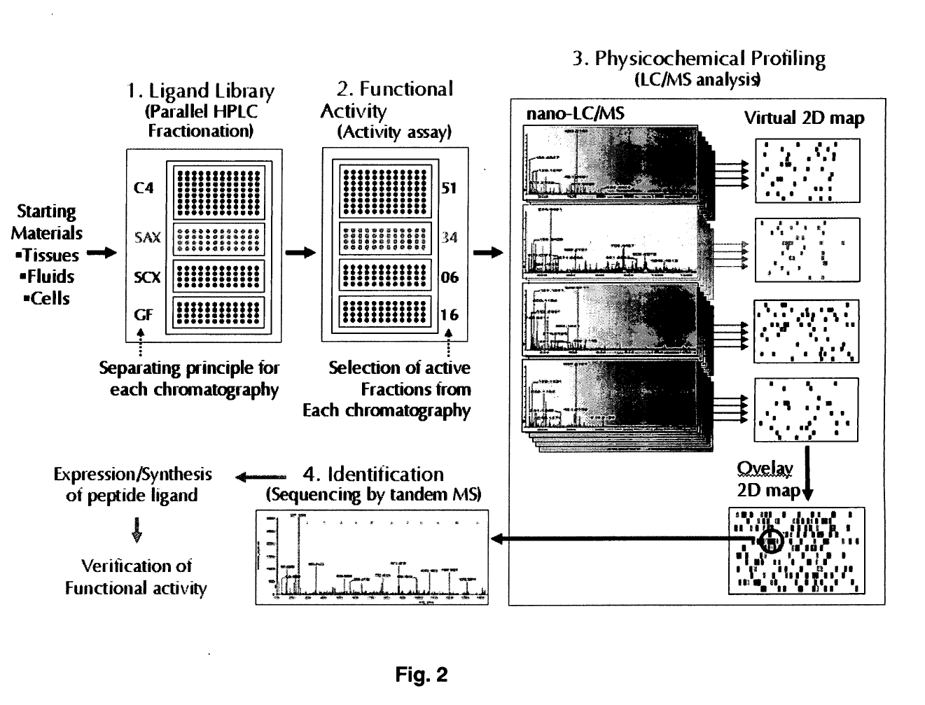

The present invention is directed to a strategy of identifying functionally novel bio-molecule agents in an efficient way. As shown in FIG. 1, the identified and profiled functionally active bio-molecule can be symbolically represented as a single dot in a multi-dimensional purification and identification assay system. Therefore, functional and physicochemical profiling of ligand library constructed by parallel column chromatographic separation may lead to the identification of novel ligands. Thus, the inventive technology may encompass various methods, including the four modules exemplified in FIG. 2, which include 1) Preparation of libraries by parallel column systems, 2) Making functional activity profiles based on various assays, 3) LC/MS analysis of the physicochemical properties of active fractions, 4) Determining the structure of the isolated agent by tandem mass analysis.

1. Preparation of Libraries from Various Sources

1) Extraction of Active Agents from Various Sources.

2) Separation of Active Agents using Various Parallel Separation Systems.

The first module consists of two steps: an extraction and a separation. Molecules of interest should be extracted from a given source. The active agents or bio-molecule agents may be various chemical compounds or peptides, proteins, carbohydrates, nucleic acids, lipids or any compound at all having any structure. Any type of extract from cells, tissue, fluids or other biological matter can be used as a starting material. Samples from eukaryotes or bacteria may be used. Plant and mammalian samples may be used. Environmental samples may be used as well, which contains the active agent of interest.

When the source and the target molecule of interest having a particular function are selected, a library including the molecule(s) of interest should be extracted considering the predicted property of the molecule of interest. The extract obtained is fractionated by parallel separation methods, such as column chromatography having different separation principles. Partially purified preparation containing the active agent of interest can also be constructed as the ligand library by further parallel chromatographic fractionation. A variety of types of separation systems, including but not limited to chromatography systems, such as column chromatography systems, and further including but not limited to a hydrophobic, ion exchange, gel filtration, or affinity column chromatography can be used for parallel processing. It is understood that a particular separation technique depends on the type of active agent that is desired to be identified having a certain type of function, and as such the number and type of separation principles as well as the apparatus used in the parallel processing may be varied according to need.

By parallel column system or parallel processing, it is understood that the processing need not occur simultaneously in time. Although such simultaneous separation procedure would be desirable, which saves time, the scope of the invention is meant to encompass using a non-sequential purification procedure not necessarily linked to time. However, it is also understood that the present invention may be practiced whereby the initial extract may be concentrated by an enrichment procedure in which the initial sample may have undergone a pre-separation process using procedures such as column chromatography, in which the initial sample that is to be parallel processed may have been run through a separation process previously. So long as the sample is parallell processed, the source of the sample is irrelevant in the practice of the invention.

Further, it is contemplated that a fraction from one parallel processed separation principle may undergo a further separation procedure in order to further purify the active agent, and the resultant fraction may form a part of the ligand library containing the active agent and such fraction is further profiled. It is also within the purview of the invention that where more than two, three, four, five or six separation principles are used in the parallel processing system, it is possible that some of the fractions may be combined further in a further separation procedure, wherein fractions obtained from the further processing may be used in the formation of the ligand library and such fractions may be further processed for profiling.

To illustrate the inventive system, if by way of example, a given starting material is fractionated efficiently by a number of 100 fractions in four different column systems, the resolution power can theoretically be estimated to be 10⁸ (100×100×100×100). In other words, one molecule out of a mixture of 100,000,000 different bio-molecules can be represented as a unique code produced by the combination of fraction number from each column. That is, if a given starting material is separately chromatographed upon four different column systems and the molecule of interest is found in fraction number 9 in hydrophobic column, fraction number 27 in anion-exchange column, fraction number 65 in cation exchange column, and fraction number 35 in gel filtration column, the specific bio-molecule can be expressed as a unique eight digit code such as 09-27-65-35.

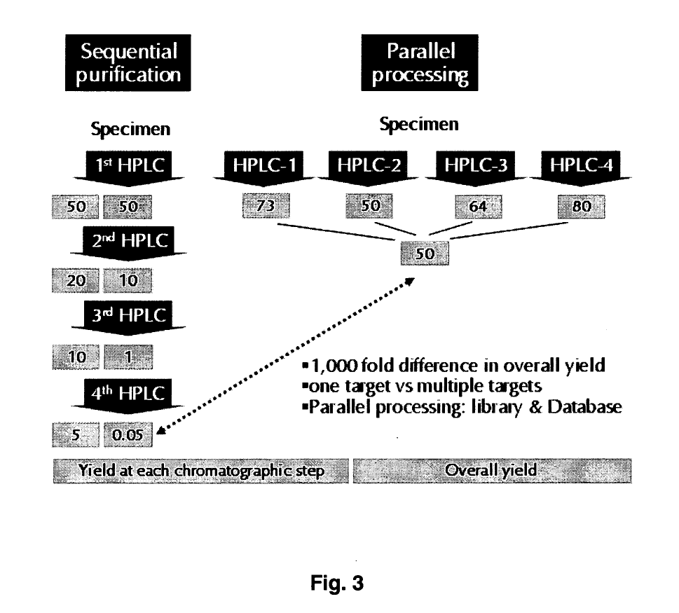

Many new ligands including peptides and proteins have been identified by traditional sequential column chromatographies. One of the limitations of the sequential purification method is low yield after each column step. As illustrated in FIG. 3, the loss of activity becomes more detrimental in late stage of sequential column chromatographies. Therefore, the problems in identifying very rare or very small active agents or biological ligands can be solved by the accommodation of the concept of parallel processing system. The good yield resulting from the parallel processing method is very helpful to identify a molecule, which may be difficult to purify using classical purification approach. In addition, the mass analysis method using without limitation mass spectrometers described infra may distinguish a molecule of interest from a mixture of materials. Finally, the combination of accurate mass analysis or mass spectrometer technology with the above-described parallel processing method synergizes the power to identify and characterize new molecule from complex mixtures.

2. Functional Activity Profile

1) Assay Method to Detect a Molecule of Interest.

2) Select Fractions Showing same Functional Activity Profile from each Separation Principle Apparatus such as Column Chromatography System.

It is necessary to establish the assay system to detect a molecule of interest. Various assay methods may be used to monitor the change affected by a molecule of interest at various levels in for example, DNA, RNA, protein, and at the cell level. Examples of such assays include without limitation, thymidine incorporation at the DNA level; RT-PCR at the RNA level; western blotting, calcium mobilization, reporter gene assay, fluorescence resonance energy transfer (FRET), pH change and c-AMP concentration measurement at the protein level; microscopy, morphology, cytotoxic assay and any physiological changes at the cell level.

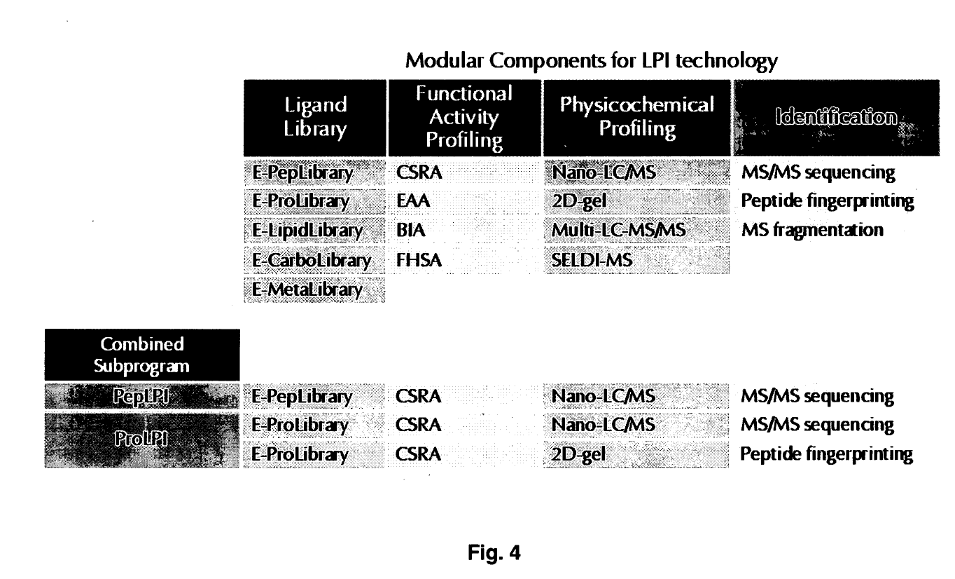

Some portions of each fraction from each column chromatography may be used for any assay system, including those mentioned above to measure the changes induced by a molecule of interest. Several fractions may be detected by one or a plurality of assay systems, or one assay system followed by at least one other assay system may be used to distinguish a molecule of interest from others. Therefore, each fraction may show different activities from various assay methods. In other words, each fraction may have its own functional activity profile. Comparing the inventive method with one-target-at-one-purification strategy accommodated by sequential purification procedure combined with an activity assay, the inventive multi-target-from-library approach together with a functional profiling of the ligand library is more effective in identifying novel ligands or bio-molecules or agents in post-genomic era, and is especially useful for those small molecules that are generally classified as peptide rather than protein and can not be easily dealt with using current methods of 2D-electrophoresis. In addition, multi-target-from-library strategy of the present invention may be readily applied to ligands other than peptide ligands by simply combining the components of each module, for example, as shown in FIG. 4.

By small bio-molecule, it is meant any molecule that is less than about 20 kDa, or less than about 15 kDa, or less than about 10 kDa, or less than about 7.5 kDa, or less than about 5 kDa, or less than about 3 kDa, or less than about 2 kDa.

3. Mass Analysis by Mass Spectrometer

1) Mass Analysis from each Fraction Showing Same Functional Activity Profile.

2) Select Common Mass to Charge (m/z) Values.

Selected fractions showing the same functional activity profile from various column chromatographies are then subjected to mass spectrometry. Mass spectrometry is a highly accurate analytical tool for determining molecular weights and identifying chemical structures. Any mass spectrometer may be used to analyze a molecule of interest. For example, the mass spectrometer can be a Matrix-Assisted Laser Desorption/Ionization (MALDI)-Time-of Flight (TOF) mass spectrometer; an ESI ion trap mass spectrometer, FT-ICR, and so on. To increase the resolving power for a mass spectrometer, a further purification system may be set up, such as liquid chromatography (LC) column preferably in-line to the mass spectrometer. The LC, for example, may be capillary- or nano-scale columns. As shown in FIG. 2, the mass peaks in the selected fractions can be re-constructed in virtual two-dimensional map (2D-map) in which x-dimension is retention time of LC and y-dimension is m/z value. The spot commonly observed in the virtual 2D-map data of active fractions from each column chromatography is selected as a physicochemical fingerprint of the molecule of interest.

4. Determine the Identity of a Selected Molecule

The structure of the selected active agent such as bio-molecule can be identified by mass analyzer or any other instrument that is able to provide the identity of the bio-molecule. In the instance of isolating a peptide ligand from a mixture of materials, peptide ions are generated by ESI source, and the tandem MS analyzers select a common m/z species of interest. This ion is then subjected to collision-induced dissociation (CID), which induces fragmentation of the peptide into fragment ions and neutral fragments. The fragment ions are analyzed on the basis of their m/z ratio to produce a product ion spectrum. The information contained in this tandem or MS-MS spectrum permits the sequence of the peptide to be deduced. Moreover, the nature and sequence location of peptide modification also can be established from an MS-MS spectrum. The inventive method provides capability of identifying novel biological ligands.

Variations in the Ligand Profiling and Identification System

In the practice of the invention, a variety of modifications to the present inventive system may be possible, such as treating the sample with an advantageous agent so as to create a sample that has reduced diversity of bio-molecules, or is rendered easier to sequence by mass spectrometer methods. Reagents that bind or specifically inhibit certain chemical groups for instance may be used to inactivate a number of non-specific agents, and reduce the pool of viable active agents for further assay, which may result in a more sensitive assay for the active agent of interest. Various enzymes may also be used to degrade away certain contaminating polypeptides. In one particular method of excluding unwanted contaminating bio-molecules from biomolecules of interest, the peptide population may be treated with various proteases that do not inhibit the specified biological activities. Such treatment using known sequence specific proteases may itself also provide a clue as to the amino acid sequence of the isolated peptide.

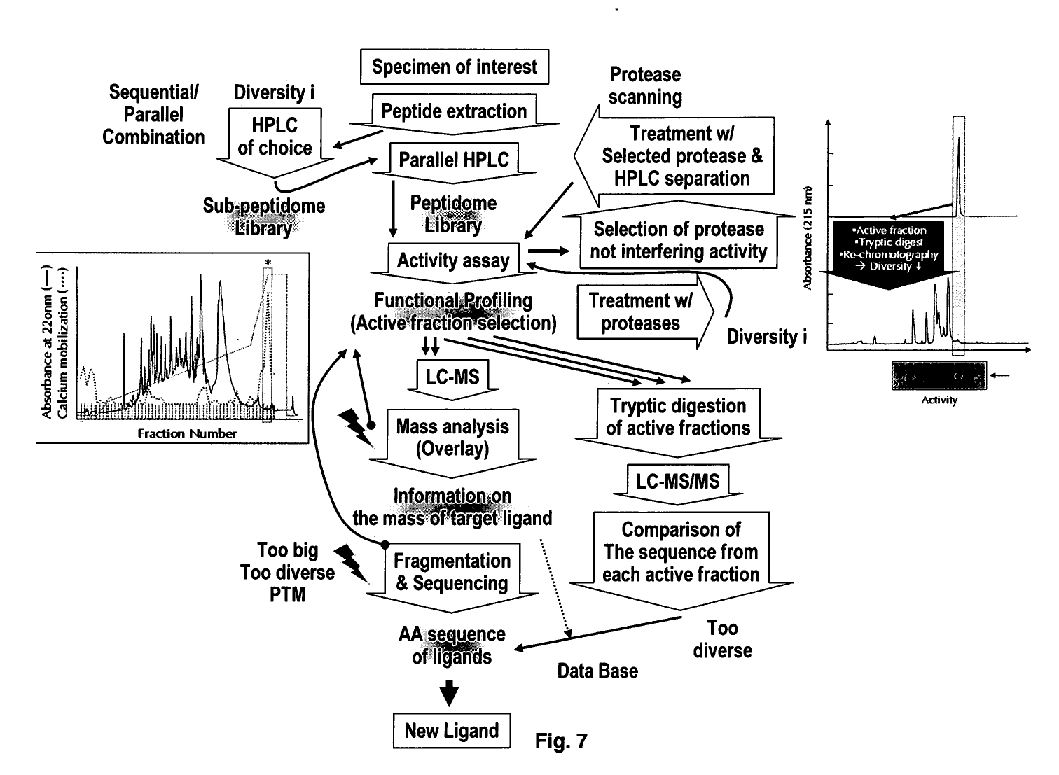

Proteinase treatment of the sample is designed to overcome the drawbacks of the complexities of the fractions to be analyzed by mass spectrometry. To this end, the combination of sequential/parallel separation step, as well as “protease scanning” procedure are interjected into the LPI technology. The enrichment of physiological activities of interest by the accomodation of the biochemical pre-fractionation can make it more possible to identify the bio-molecules, such as peptides that are responsible for the specified physiological activities. Moreover, the enzymatic or chemical cleavage of the fractions containing bio-molecules of interest followed by LC/MS-MS analysis can be utilized for the direct comparison of amino acid sequences rather than the physicochemical properties of bio-molecules such as mobility in chromatographic procedure and molecular mass (FIG. 7).

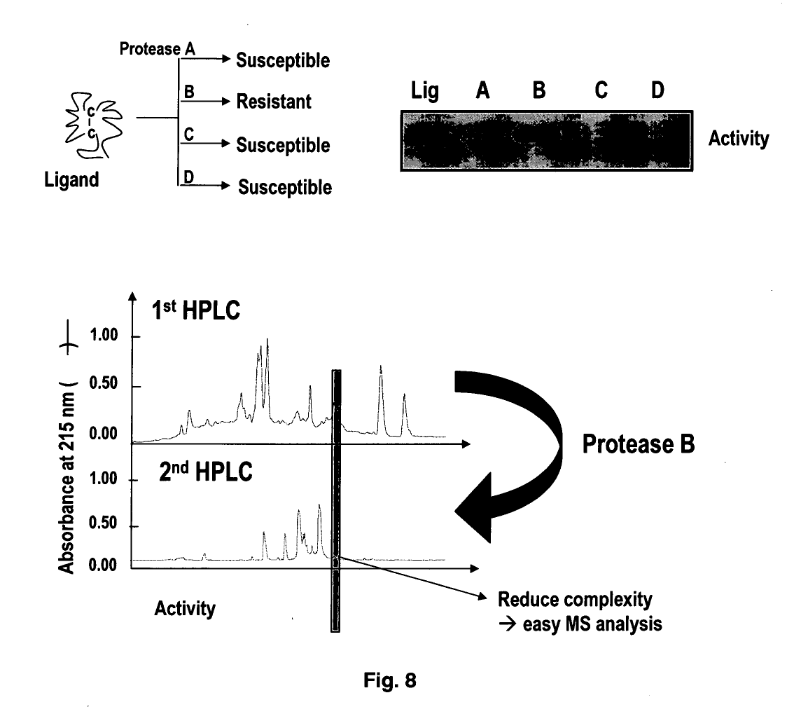

In a physical sense, peptide ligands tend to have defined and compact structures compared with more structurally relaxed contaminating protein fragments which can be artificially generated during extraction and separation procedures. By using the characteristics of ligands, a protease which cannot digest the bio-molecules of interest can be selected by treating the sample with several sets of proteases separately and monitoring the loss of activity. The treatment with the selected protease which does not inhibit the bio-molecules of interest can be efficiently applied for reducing the complexities of a given fraction by excluding other contaminants susceptible to protease treatment through column chromatographic separation (FIG. 8).

A variety of proteases or peptidases are available. Peptidases may be categorized into exopeptidases or endopeptidases. Proteinases are synonymous with endopeptidases, and are available as a sequence specific digestion of polypeptides, and to provide an identifying profile of the polypepide of interest. Proteinases may include but not limited to serine proteinase, cysteine proteinase, aspartic proteinase, and metallo proteinase. Other proteinases exist for which their catalytic mechanism is unidentified.

Briefly, serine proteinases comprise two distinct families. The chymotrypsin family which includes the mammalian enzymes such as chymotrypsin, trypsin or elastase or kallikrein and the substilisin family which include the bacterial enzymes such as subtilisin. The general 3D structure is different in the two families but they have the same active site geometry and their catalysis proceeds via the same mechanism. The serine proteinases exhibit different substrate specificities, which are related to amino acid substitutions in the various enzyme subsites interacting with the substrate residues. Some enzymes have an extended interaction site with the substrate whereas others have a specificity restricted to the P1 substrate residue. Three residues which form the catalytic triad are essential in the catalytic process i.e., His 57, Asp 102 and Ser 195 (chymotrypsinogen numbering).

The cysteine proteinases include the plant proteases such as papain, actinidin or bromelain, several mammalian lysosomal cathepsins, the cytosolic calpains (calcium-activated) as well as several parasitic proteases (e.g Trypanosoma, Schistosoma). Papain is the archetype and the best-studied member of the family. Like the serine proteinases, catalysis proceeds through the formation of a covalent intermediate and involves a cysteine and a histidine residue. The essential Cys25 and His159 (papain numbering) play the same role as Ser195 and His57 respectively.

Most of the aspartic proteinases belong to the pepsin family. The pepsin family includes digestive enzymes such as pepsin and chymosin as well as lysosomal cathepsins D and processing enzymes such as renin, and certain fungal proteases (penicillopepsin, rhizopuspepsin, endothiapepsin). A second family comprises viral proteinases such as the protease from the AIDS virus (HIV) also called retropepsin.

The metallo proteinases are found in bacteria, fungi as well as in higher organisms. They differ widely in their sequences and their structures but the great majority of enzymes contain a zinc atom, which is catalytically active. In some cases, zinc may be replaced by another metal such as cobalt or nickel without loss of the activity. Bacterial thermolysin has been well characterized and its crystallographic structure indicates that zinc is bound by two histidines and one glutamic acid. Other families exhibit a distinct mode of binding of the Zn atom.

In the practice of the invention, such proteinases may be applied to the peptide extract prior to or after parallel processing. Preferably, the proteinase may not interfere with the assay, and as such, the proteinases should be prescreened so that they do not inhibit or provide artifactual results in the assay.

The chromatographic separation following the treatment of the active fractions with a proteinase such as trypsin which do not interfere with the specified activity may result in the efficient separation of trypsin-resistant bio-molecules of interest from trypsin-susceptible contaminating proteins. The non-proteinase treated active fraction may undergo physicochemical profiling for example so that a virtual 2-D image of the various peptide ligands is presented. In comparison, the active fractions that have been subjected to proteinase digestion and further separated to form an active fraction may also produce virtual 2-D image, whereby the sequence of the peptide ligand from both non-proteinase digested and proteinase digested portions may be provided through sequencing protocol through an instrument such as tandem mass spectrometer. (FIGS. 8).

Instructions

The present invention is also directed to instructions regarding the use of the inventive ligand profiling and identifying system and method. Such instructions may be in a permanent or temporary format. The instructions may be in written form, such as but not limited to an operating manual. Such instructions may be in relation to a new compound screening method or drug discovery method. The instructions may be via a computer screen via cathode ray tube, LCD, LED, and so on, so long as the instructions are visible through the eye. The instructions may also be in the form of audio/visual media.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

Peptides or proteins are the major groups of biological ligands of receptors identified so far. Especially, around 75% of these total polypeptide ligands have a molecular mass less than 20 kDa. These peptide ligands have been identified through biochemical separation and purification from various source organisms. There exist many difficulties in analyzing the peptide ligands by homology-based search through genomic approach, because the ligands have small molecular weight and are further post-translationally modified. Recently, it has been possible, by the development of both mass analysis technology and high throughput screening technology, to characterize the identity of the peptide ligands present in small amount and to screen rapidly and efficiently the ligands affecting various kinds of cellular activities with various means. In this respect, the concept of ligand proteomics has occurred by which ligands working in the extracellular space have been analyzed through integration of the independent processes for both screening of ligand library and their identification. In this study, we established a new strategy to identify a new peptide ligand and identified an insulin-like peptide which affects cellular glucose metabolism. This hormone may be used like insulin as biomedicine or may be used as a new marker to diagnose or treat diabetes.

Recently, various peptide hormones related to metabolism of both glucose and lipid have been identified from adipocyte, intestine, and brain as well as islets of Langerhans, suggesting that a body organizes multilayer network of metabolic control through intercellular communication. The identification of new hormone, therefore, provides both the understanding of various mechanisms of energy balance and the idea of diagnosis and treatment for diabetes.

Insulin is a well-known regulator of virtually all aspects of adipocyte biology. The initial molecular signal for insulin action involves the activation of the insulin receptor tyrosine kinase, which results in phosphorylation of insulin receptor substrates (IRSs) on multiple tyrosine residues. The phosphorylation of IRS stimulates the mitogen-activated protein (MAP) kinase (ERK). As another pathway of the insulin signal, AKT can be activated through the phosphatidylinositol (PI) 3-kinase.

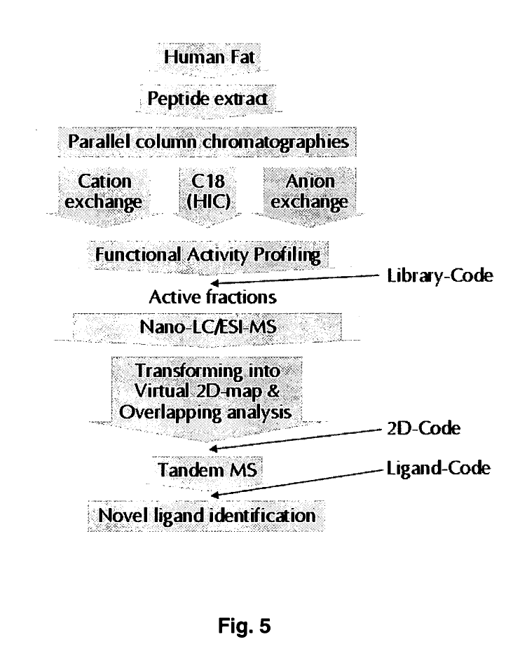

The present inventive ligand profiling and identification system has been used to identify new endogenous peptide ligands from human fat. A detailed flow chart is shown at FIG. 5. Peptides are extracted from fat and separated into many fractions by using three different HPLC column chromatographies, thereby making a ligand library. All of the fractions are assayed by western blotting to screen for molecules triggering the phosphorylation of the proteins in insulin signaling network. The fractions from each column chromatography displaying the same functional activity profile were selected, and unique code (so called Library-Code) was given for the molecule of interest. Active fractions from each column chromatography were separately analyzed by nano-LC/MS, and data were transformed into three different virtual 2D-maps. The molecules represented as being at the same location in 2D-maps (2D-Code) were selected by overlapping the virtual 2D-maps obtained from the analyses of the active fractions. Three 2-D maps were overlaid and two common mass contained in three maps were selected. Finally, two candidate Ligand-Codes (the unique code produced by the combination of Library-Code and 2D-Code) were obtained. The selected masses are analyzed by LC-MS-MS to characterize their identities.

I. Ligand library

1) Preparation of Peptide

Human abdominal fat of an obese patient was obtained from Yeungnam University Medical Center. After mixing with the same volume of 1×PBS, the fat was homogenized by mixer and centrifuged at 3,000 rpm for 15 min at 4° C. The supernatant was collected and the precipitant was added by same volume of 1× PBS. The homogenization step above for the precipitant was repeated to maximize extraction of crude peptides. The extracted peptides from each extraction step were pooled, mixed with 10× volume of 70% (v/v) acetone, 1 M acetic acid, and 20 mM HCl and centrifuged at 8,000 rpm for 30 min at 4° C. The supernatant was extracted three times with diethyl ether. The aqueous phase was centrifuged at 12,000× g for 5 min at 4° C., and the supernatant was loaded on five 1 g HLB cartridges. Cartridges were washed with 5% CH₃CN/0.1% TFA, and then eluted with 50% CH₃CN/0.1% TFA. The eluate was lyophilized and dissolved in 1M acetic acid.

2) Separation of Crude Peptides by HPLC System

For making a Ligand library, we used three different-HPLC systems including C18, cation exchange and anion exchange chromatographies which have different principles for separation of proteins/peptides.

(A) A part of the extracted peptide library from human fat was separated by C18 reverse-phase HPLC. A column (Vydac 218 TP510; 10 mm X 250 mm) was equilibrated with water/0.1% TFA. A linear gradient was started from 0% to 15% of acetonitrile in 15 min, followed by 45% for 30 min and followed by 50% for 20 min at a flow rate of 4 ml/min at room temperature. 4-ml fractions were collected and 1% of each fraction was used for western blotting. Acetonitrile gradient and absorbance of eluted materials are indicated by solid and dotted lines, respectively. There is a peak eluted at 35 min showing activities for pIRS proteins.

(B) The extract was loaded onto a column of Mono S 5/5 (5 mm×50 mm) equilibrated with 5 mM sodium phosphate/0.25% CH₃CN pH 3.0 at flow rate of 1 ml/min at room temperature. 500-ul fractions were collected, and 2% of each fraction was set aside and assayed by western blot described below with anti-pIRS antibody as a secondary antibody to find IRS phosphorylation in C2C12 cells. There is a peak eluted at 19 min showing activities for pIRS proteins.

(C) A column of Mono Q 5/5 (5 mm×50 mm) was equilibrated with 20 mM NH₄HCO₃, pH 7.9. Some portion of crude extract of fat was applied to this column at flow rate of 1 ml/min at room temperature. 500-ul fractions were collected and 2% of each fraction was used for western blotting as described. There is a peak eluted at 13 min showing activities for pIRS proteins.

The peptide library was fractionated efficiently by the number of about 96 fractions from C18 column and about 48 fractions from the remaining two columns (cation exchange and anion exchange). Therefore, the resolution power could be 221,184 (96×48×48) if all of peptides have separated evenly. One peptide of interest from about 10⁵ of different peptides in a peptide library should be selected. To our knowledge, the exact number of peptides from human fat used has not been reported. When we calculate the number of peptides present in one fraction from C18 column, the number is about 700. It is calculated that the total number of peptides from human fat is about 7×10⁴ (700×96) which is enough to be analyzed by our system.

II. Functional Activity Assay

To identify the ligand of interest, it is necessary to establish specific functional assay system. We have first determined to find new insulin-like ligands. One of the early downstream target of insulin signal is the insulin receptor substrate (IRS). As a major gate of insulin pathway, IRS should be phosphorylated when insulin or other ligands outside of cell give a signal to cell, finally controlling various cellular events inside the cell such as glucose uptake via GLUT4. Western blot analysis is applied to check the level of p-IRS as described below for each fraction from HPLCs.

A detailed method of western blotting is as follows. After growing in DMEM containing 10% FBS, C2C12 cells were plated on 24-well plates (5×10⁴ cell/well) and changed to serum-free medium for 24 hrs. Cells were treated with each aliquot from HPLC fractions for 5 min and the reactions were terminated by the addition of 5× sample buffer (150 mM Tris HCl, pH6.8, 12% SDS, 25% glycerol, 12.5% 2-mercaptoethanol, 0.05% bromophenol blue), lysed by intermittently vortexing for 30 min. After transferring the samples to eppendorf tubes, the lysates was boiled for 3 min and centrifuged for 2 min. Aliquots of these supernatants were separated through 8% SDS-PAGE, and the proteins were transferred to nitrocellulose at 90V for 90 min. After the transfer, the membrane was blocked with 5% skim milk in TTBS (10 mM Tris HCl, pH 7.6, 150 mM NaCl, 0.05% Tween 20), and the membrane was incubated at room temperature for 3 hrs with primary antibodies in TTBS. After six washes with TTBS, 1 hr of incubation with horseradish peroxidase-conjugated anti-pIRS secondary antibody (anti-rabbit) was performed and signal was detected with enhanced chemiluminescence (Amersham).

We found one region showing an increase of pIRS from fractions of three column chromatographies, respectively. We collected active fractions from three different columns and dried out for the next mass analysis.

III. Analysis of Mass and Determination of the Sequence of a Peptide

There are many different types of mass spectrometers including generally MALDI-TOF and ESI mass spectrometer. Recently, liquid chromatography (LC) column is set up in front of mass spectrometer to detect many m/z peaks from complex mixtures. We also connected nano-LC into ESI mass spectrometer to further separate mixtures and concentrate peptides within a short retention time. Below is a detailed explanation regarding nano-LC and mass spectrometer.

Nano-LC

One tenth of one assay amount showing p-IRS activity has been used for analysis by nano-LC-MS. For nanoscale LC, experiments were performed with an UltiMate Nano LC system (LC Packings-A Dionex Company, Amsterdam, Netherlands) equipped with FAMOS autosampler and Swichos (LC Packings-A Dionex Company). Lyophilized peptide was reconstituted in 5% (v/v) aqueous acetic acid. Acetic acid was used to help peptide solubilization, and aliquots were kept in the freezer at −20° C. The loading solvent consisted of a 1% (v/v) acetic acid aqueous solution delivered at a flow rate of 25 ul/min. Sample preconcentration and desalting was performed with a LC pump that was operated isocratically at a flow-rate of 25 ul/min. Cartridge type precolumns (LC Packings-A Dionex Company) with a length of 5 mm and an ID of 300 um were used to preconcentrate and desalt samples. The preconcentration column was filled with a Sum, 100A C18 PepMap™ stationary phase (LC Packings-A Dionex Company).

The chromatography was performed on an analytical fused-silica nanocolumn of 15 cm×75 um I.D. packed with a C18 PepMap (3 um) stationary phase (LC Packings-A Dionex Company). The aqueous mobile phase (A) contained 0.1% (v/v) formic acid solution, and the organic mobile phase (B) contained 0.1% (v/v) formic acid in acetonitrile. A linear gradient started from 10% to 80% of acetonitrile in 50 min followed by a washing step of 80% of acetonitrile for 20 min. Finally, the column was re-equilibrated with the initial mobile phase for 20 min. A flow rate of 200 nl/min was delivered throughout the entire procedure. The UltiChrom software suite (LC Packings-A Dionex Company) was used for instrument control.

2) Mass Spectrometer

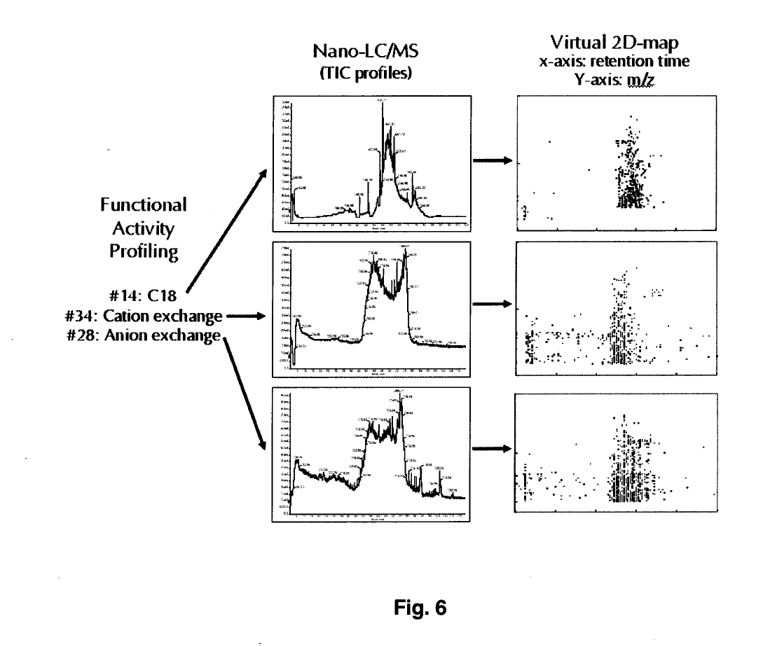

Measurements were carried out in the positive electrospray ionization mode and were performed on a QSTAR PULSAR I hybrid Q-TOF MS (Appliedbiosystems/PE SCIEX, Toronto, Ontario) equipped with a nanospray source. The QSTAR was operated at 8,000-10,000 resolution with a mass accuracy of 10-30 ppm using an external calibration maintained for 24 hrs. Coupling nano-LC to MS was achieved by PRO-ADP2 assembly (New Objective, Cambridge, Mass., USA) acting as a spray tip mount, which was mounted on the xyz-stage of the mass spectrometer. A distally coated fused silica Pico tip (New Objective, Cambridge, Mass., USA) (360 um OD/20 um ID/10 um tip ID) was used as a nanospray needle. The tip was positioned approximately 3 mm from curtain plate. The voltage of spray tip was set at 1,400-1,600V. FIG. 6 shows mass results expressed with total ion count (TIC) [y-axis] against retention time [x-axis] of active fractions showing IRS-phosphorylation activity by nano LC/MS and the transformation of TIC data into 2D-maps. The three 2-D maps corresponding to the same peptide are superimposed and then two common masses, 3402 and 4818 of average mass, are selected. The average mass of 3402 comes up starting at 54 min of nano-LC column for 1 min at all three different active fractions. The other 4818 is shown starting at 67 min for several minutes. These two masses are sequenced by using tandem mass spectrometry.

We have discovered two mass-candidates which may represent an active ligand allowing the phosphorylation of IRS protein by the inventive technology. As described above, this technology is useful to identify a specific molecule from various mixtures without requiring a final purification step.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method of identifying an active agent in a sample comprising subjecting the sample to a plurality of separation principles in parallel, obtaining an active fraction from each separation principle, profiling physiochemical properties of the active fraction so as to obtain the agent that is common in each of said fraction, wherein said agent is identified.

2. The method according to claim 1, wherein the sample is a mammalian extracellular fluids.

3. The method according to claim 1, wherein the sample is a mammalian cell/organ extract.

4. The method according to claim 1, wherein the sample is a plant cell/organ extract.

5. The method according to claim 1, wherein the sample is an environmental sample.

6. The method according to claim 1, wherein the agent is a bio-molecule.

7. The method according to claim 6, wherein the bio-molecule is a polypeptide, carbohydrate, lipid or nucleic acid.

8. The method according to claim 7, wherein the bio-molecule is a peptide.

9. The method according to claim 6, wherein the bio-molecule specifically binds to a G protein coupled receptor.

10. The method according to claim 6, wherein the bio-molecule specifically binds to a growth factor receptor.

11. The method according to claim 6, wherein the bio-molecule specifically binds to an adhesion molecule.

12. The method according to claim 6, wherein the bio-molecule is a protease.

13. The method according to claim 1, wherein the agent is less than about 20 kDa.

14. The method according to claim 13, wherein the agent is less than about 10 kDa.

15. The method according to claim 14, wherein the agent is less than about 2 kDa.

16. The method according to claim 1, wherein at least one of the separation principles is in the form of column chromatography.

17. The method according to claim 16, wherein the chromatography is hydrophobic interaction chromatography, cation exchange chromatography, anion exchange chromatography, gel permeation chromatography, or affinity chromatography.

18. The method according to claim 1, wherein said profiling physiochemical properties comprises passing the active fraction through a liquid chromatography (LC) column.

19. The method according to claim 18, wherein the liquid chromatography column is nano-LC column.

20. The method according to claim 18, wherein the LC column is connected to a mass spectrometer.

21. The method according to claim 1, wherein the active fraction is determined by cell based assay.

22. Instructions comprising the method according to claim 1.

23. A method of assigning a code to an active agent containing fraction in a sample comprising subjecting the sample to a plurality of separation principles in parallel, obtaining an active fraction from each separation principle, assigning a first unique identifying code to the active fraction, and gathering and combining the identifying code to form a second unique assigning code for the active agent containing fraction.
